(19) United States
(12) Patent Application Publication
(10) Pub. No.: US 2015/0313952 A1
(43) Pub. Date: Nov. 5, 2015

(54) SYNBIOTIC COMPOSITION AND USE THEREOF

(71) Applicant: NESTEC S.A., Vevey (CH)

(72) Inventors: Dantong Wang, Prilly (CH); Christelle Schaffer-Lequart, Mezieres (CH); Jalil Benyacoub, Epalinges (CH); Pascal Volery, Bulle (CH); Jean-Yves Chuat, Prilly (CH)

(21) Appl. No.: 14/646,139

(22) PCT Filed: Nov. 29, 2013
(86) PCT No.: PCT/EP2013/075157

§ 371 Date: May 20, 2015

(30) Foreign Application Priority Data
Nov. 29, 2012 (EP) 12194906.9

(51) Int. Cl. A61K 35/745; A23L 1/30; A61K 31/716

(52) U.S. Cl. CPC A61K 35/745; A61K 31/716; A23L 1/3014; A61K 2035/115

(57) ABSTRACT

The invention relates to a synbiotic composition, its use for use the establishment and/or the maintenance of a healthy gut environment, as well as food compositions comprising said synbiotic composition.

### TECHNICAL FIELD

The invention also relates to a synbiotic composition for use in the establishment and/or the maintenance of a healthy gut environment.

### BACKGROUND OF THE INVENTION

Any discussion of the prior art throughout the specification should in no way be considered as an admission that such prior art is widely known or forms part of common general knowledge in the field.

Arabino-xylo-oligosaccharides (AXOS) are a prebiotic ingredient. Several authors have shown that AXOS improves digestive health (Broekaert et al., 2011; Francois et al., 2012; Grootaert et al., 2009). WO 2009/040445 A2 mentions the use of oligosaccharides derived from arabinoxylan in the prevention and treatment of gastrointestinal infection of an animal or human being with bacteria associated with gastroenteritis. Although AXOS-related products have been found to increase the level of immunopotentiating activity (Ogawa et al., 2005) and ameliorate inflammation in colitis (Komiyama et al., 2011), the effect of AXOS on immune function is still largely unknown beside one study that showed its inhibition on the colonization of Salmonella in an animal model (Eeckhaut et al., 2008).

WO 2010/066012 A2 describes nutritional compositions enriched with arabinoxlan-oligosaccharides and further comprising either or both water-unextractable arabinoxylans or water-soluble arabinoxylans, preferably both.

WO 2009/117790 A2 describes an (arabino)xylan oligosaccharide preparation. WO 2010/088744 A2 describes a method for the extraction and isolation of solubilised arabinoxylan depolymerisation products, such as soluble arabinoxylan, arabinoxylan-oligosaccharides, xylose and arabinose.

Crittenden et al. (2001) propose in vitro screening procedures that can be used to integrate complementary probiotic and prebiotic ingredients for new synbiotic functional food products. They employed this procedure to select a probiotic Bifidobacterium strain to complement resistant starch (Hi-maize™) in a synbiotic yoghurt.

WO 2008/071930 A1 discloses a composition comprising one or more live Bifidobacterium lactis strains and a saccharide component comprising xylo-oligosaccharides with a degree of polymerisation of from 2 to 100.

WO 2006/002495 A1 discloses a food or beverage comprising arabinoxylans such as AXOS and Bifidobacterium or Lactobacillus.

WO 2101/020639 A1 discloses an arabinoxylan preparation for modulating the barrier function of the intestinal surface, primarily by modulating the mucosa-associated microbial community towards a relative increase of health beneficial bacteria. The preparation my comprise probiotics such as Bifidobacterium or Lactobacillus.

WO 00/33854 A1 discloses to a preparation having a health-promoting action, which contains one or more probiotics and one or more non-digestible oligosaccharides. The probiotics may be chosen from strains of Lactobacillus, Bifidobacterium, or Saccharomyces.

WO 02/051264 discloses a nutritional composition having a beneficial effect in the gastrointestinal tract, especially anti-adhesion effect, on pathogenic micro-organisms, and a bifidogenic effect. The composition contains non-digestible oligosaccharides.

### SUMMARY OF THE INVENTION

The inventors have found that a combination of i) a probiotic micro-organism comprising a Bifidobacterium strain, and (ii) an oligosaccharide component comprising arabino-xylo-oligosaccharides, has a synergistic effect on the establishment and/or the maintenance of a healthy gut environment.

Therefore, it is desirable to propose food products comprising this synbiotic composition, for use in the establishment and/or the maintenance of a healthy gut environment, as an improvement over, or at least an alternative to, the prior art.

To this end, an embodiment of the invention proposes a synbiotic composition for use in the establishment and/or the maintenance of a healthy gut environment, wherein said composition comprises (i) a probiotic micro-organism comprising a Bifidobacterium strain, and (ii) an oligosaccharide component comprising: from 30 to 40% by weight of arabino-xylo-oligosaccharides having an average degree of polymerization from 2 to 50, and from 35 to 45% by weight of xylo-oligosaccharides having a degree of polymerization ranging from 2 to 9. Establishment and/or maintenance of a healthy gut environment may comprise modulation of the acidity in the colon, modulation of the short-chain fatty acids concentration in the colon, and modulation of the gut microflora.

Another embodiment of the invention proposes a synbiotic composition comprising (i) a probiotic micro-organism comprising a Bifidobacterium strain, and (ii) an oligosaccharide component comprising arabino-xylo-oligosaccharides.

Another embodiment of the invention proposes a food composition comprising said synbiotic composition.

In a further embodiment of the invention, said food composition is for use in the establishment and/or the maintenance of a healthy gut environment. Establishment and/or maintenance of a healthy gut environment may comprise modulation of the acidity in the colon, modulation of the short-chain fatty acids concentration in the colon, and modulation of the gut microflora.

In an embodiment of the invention, the food composition comprises from 100 mg to 10 g of arabino-xylo-oligosaccharides component per daily dose, and/or from 10̂6 to 10̂12 cfu of Bifidobacterium per gram of food composition.

In embodiments of the invention, said Bifidobacterium strain may be selected from Bifidobacterium longum strains, Bifidobacterium lactis strains, Bifidobacterium animalis strains, Bifidobacterium breve strains, Bifidobacterium infantis strains, Bifidobacterium adolescentis strains, and mixtures thereof. In embodiments of the invention, said Bifidobacterium strain may be selected from Bifidobacterium longum NCC 3001, Bifidobacterium longum NCC 2705, Bifidobacterium breve NCC 2950, Bifidobacterium lactis NCC 2818, and mixtures thereof. Preferably said Bifidobacterium strain is a Bifidobacterium lactis strain. Preferably said probiotic micro-organism consists essentially of Bifidobacterium lactis NCC 2818.

In embodiments of the invention, said arabino-xylo-oligosaccharides (AXOS) has an average degree of polymerisation (DP) comprised between 3 and 8, and an arabinose to xylose ratio (A/X ratio) comprised between 0.18 to 0.30. In embodiments of the invention, a ferulic acid residue is bound to said arabino-xylo-oligosaccharides (AXOS), via an ester linkage, preferably to an arabinose residue. In embodiments of the invention, said oligosaccharide component further comprises beta-glucan, xylo-oligosaccharides, xylobiose, and mixtures thereof.

These and other aspects, features and advantages of the invention will become more apparent to those skilled in the art from the detailed description of embodiments of the invention, in connection with the attached drawings.

### BRIEF DESCRIPTION OF THE DRAWINGS

FIG. 1 shows the effect of 10 μg/ml AXOS on chemokine secretion in a Caco-2/PBMC (peripheral blood mononuclear cell) co-culture system over 24 hours (Example 1).

FIG. 2 shows the effect of 10 μg/ml AXOS with 10̂7 CFU/ml Bifidobacterium lactis NCC 2818 on chemokine secretion in a Caco-2/PBMC co-culture system over 24 hours (Example 1).

FIGS. 3, 4 and 5 show the evolution of the short-chain fatty acids (SCFA) concentrations in the ascending (AC), transverse (TC) and descending colon (DC), for diets PRE (FIG. 3), PRO (FIG. 4) and SYN (FIG. 5) respectively, in the experimental setup of Example 2. Triangle: butyrate; Squares: propionate; Diamonds: acetate; Cross: total SCFA. C1, C2: control weeks 1 and 2. T1, T2, T3: test weeks 1, 2 and 3. PRE: AXOS 2.5 g/day; PRO: B. lactis 2.8×10⁹ CFU per day; SYN: AXOS 2.5 g/day and 2.8×10⁹ CFU per day.

FIGS. 6, 7 and 8 show the consumption of NaOH (N) and HCl (H) in the ascending (AC), transverse (TR), and descending (DC) colon throughout the course of the experiment described in Example 2, for diets PRE (FIG. 6), PRO (FIG. 7) and SYN (FIG. 8) respectively. PRE: AXOS 2.5 g/day; PRO: B. lactis 2.8×10⁹ CFU per day; SYN: AXOS 2.5 g/day and 2.8×10⁹ CFU per day.

FIG. 9 shows the cumulative number of B. lactis 16S copies over the 3-week test periods in the colonic compartments (ascending AC, transverse TC and descending DC colon) for the three diets PRE, PRO and SYN, in the experimental set-up of Example 2. PRE: AXOS 2.5 g/day; PRO: B. lactis 2.8×10⁹ CFU per day; SYN: AXOS 2.5 g/day and 2.8×10⁹ CFU per day.

FIG. 10 shows the cumulative total Bifidobacteria population over the 3-week test period relative to the control week populations, in the colonic compartments (ascending AC, transverse TC and descending DC colon) for the three diets PRE, PRO and SYN, in the experimental set-up of Example 2. PRE: AXOS 2.5 g/day; PRO: B. lactis 2.8×10⁹ CFU per day; SYN: AXOS 2.5 g/day and 2.8×10⁹ CFU per day.

FIGS. 11 and 12 show the effect of B. lactis (10̂7 CFU/mL) alone, AXOS (100 mg/mL) alone, and a combination of B. lactis (10̂7 CFU/mL) and AXOS (100 mg/mL), on the invasion of CaCo2 cells by Salmonella, with differentiated Caco-2 cells (FIG. 11) and differentiated polarised Caco-2 cells (FIG. 12) (Example 4).

### DETAILED DESCRIPTION OF THE INVENTION

Unless the context clearly requires otherwise, throughout the specification, the words “comprise”, “comprising” and the like are to be construed in an inclusive sense, that is to say, in the sense of “including, but not limited to”, as opposed to an exclusive or exhaustive sense.

Unless noted otherwise, all percentages in the specification refer to weight percent, where applicable.

Unless defined otherwise, all technical and scientific terms have and should be given the same meaning as commonly understood by one of ordinary skill in the art to which this invention belongs.

The term “probiotic” is defined as live micro-organisms that, when administered in adequate amounts, confer health benefits to the host (FAO/WHO Guidelines). As mentioned above, the probiotic micro-organism is preferably a Bifidobacterium strain selected from Bifidobacterium longum strain, Bifidobacterium lactis strain, Bifidobacterium animalis strain, Bifidobacterium breve strain, Bifidobacterium infantis strain, Bifidobacterium adolescentis strain, and mixtures thereof. For instance, said Bifidobacterium strain is selected from Bifidobacterium longum NCC 3001, Bifidobacterium longum NCC 2705, Bifidobacterium breve NCC 2950, Bifidobacterium lactis NCC 2818, and mixtures thereof.

The following strains were deposited under the Budapest treaty at the Collection Nationale de Cultures de Micro-Organismes (CNCM, Institut Pasteur, 28 rue du Dr Roux, 75724 Paris Cedex 15 France):

| Strain | Accession number | Deposit date |
|---|---|---|
| Bifidobacterium longum | CNCM I-2618 | 29 Jan. 2001 |
| NCC 2705 | | |
| Bifidobacterium breve NCC 2950 | CNCM I-3865 | 15 Nov. 2007 |
| Bifidobacterium lactis NCC 2818 | CNCM I-3446 | 7 Jun. 2005 |

Bifidobacterium longum NCC 3001 was deposited by Morinaga, at the American Type Culture Collection (ATCC), under accession number ATCC BAA-999. It is publicly available, as shown for instance by the abstract PG3-11 by Mercenier et al. (2006).

The probiotic micro-organism can be provided as live probiotics, or in an inactivated state. Inactivated probiotic micro-organisms are described, for instance, in WO 2010/130659, WO 2010/130660, or WO 2011/000621.

“Prebiotics” are compounds, usually oligosaccharides, that cannot be digested by enzymes of the upper gastro-intestinal tract but are fermented selectively by some types of intestinal bacteria in the colon, or large intestine.

A “synbiotic” is the synergistic combination of a probiotic component and a prebiotic component. A synergy can be observed when the combined effect of two treatments, components, or ingredients, is different from the purely additive effect that can be expected from each treatment, component, or ingredient taken separately. Usually, the effect of the combination is greater than the added effect of each treatment, component, or ingredient taken separately. The mix of a prebiotic and a probiotic is not always a synbiotic. This is the case when no synergistic effect is observed.

“Arabino-xylo-oligosaccharide” or “AXOS” are oligosaccharides consisting of a backbone of xylose residues linked together via β-(1-4) osidic linkages, where at least one xylose residue is substituted with one or two arabinose units at the O-2, the O-3, or both the O-2 and O-3 positions of xylose residues. In an embodiment, AXOS have an average degree of polymerisation (DP) between 2 and 50, preferably from 2 to 15, and even more preferably from 2 to 8. The lower DP value of AXOS can be as low as 2, 3 or 4. The higher DP value of AXOS can be up to 50, 40, 30, 20, 15, 10, 9, 8, 7 or 6. In an embodiment, AXOS have an arabinose to xylose ratio (A/X ratio), also referred to as the average degree of arabinose substitution, comprised between as low 0.18 or 0.19, and up to 0.30, 0.27, 0.24, or 0.21. In an embodiment, AXOS have an average DP between 3 and 8, and an A/X ratio comprised between 0.18 to 0.30. Minimum and maximum values mentioned above can be combined.

Preferably, the oligosaccharide component comprises a mixture of xylo-oligosaccharides (XOS), AXOS, and optionally, other carbohydrates which may be found in the starting material used to prepare said oligosaccharide component. XOS are xylose oligomers having a degree of polymerization of 2 to 9. Preferably, xylobiose (XOS have a DP of 2, also noted as X₂) represents from 15% by weight to 25% by weight of the dry matter of the oligosaccharide component. Preferably, XOS having a DP from 2 to 9 (X₂₋₉) represent from 35% by weight to 45% by weight of the dry matter of the oligosaccharide component. Preferably, AXOS represent from 30% by weight to 40% by weight of the dry matter of the oligosaccharide component. In an embodiment, ferulic acid residues may be linked to arabinose residues of the arabinoxylo-oligosaccharides via an ester linkage.

In an embodiment, the oligosaccharide component comprises 35 to 45% by weight of xylo-oligosaccharides having a degree of polymerization ranging from 2 to 9, and 30 to 40% by weight of AXOS having an average degree of polymerization from 2 to 50, preferably from 2 to 15, and more preferably from 3 to 8. In an embodiment, said AXOS has an A/X ratio comprised between 0.18 to 0.30. In an embodiment, xylobiose represents from 15 to 25% by weight of the oligosaccharide component.

Preferably, said oligosaccharide component, and especially said arabino-xylo-oligosaccharides, derives from cereals, preferably selected from wheat, rice, maize, oats, barley, sorghum, rye.

The synbiotic composition can be incorporated into a food composition, for instance by dry mixing the components of the synbiotic composition successively, together or as a premix, into a food composition, following regular processing techniques. In an embodiment, such food compositions comprise from 100 mg to 10 g of oligosaccharide component per daily dose. In another embodiment, such food compositions comprise from 10̂6 to 10̂12 cfu of a Bifidobacterium strain per gram of food composition. In another embodiment, such food compositions comprise from 100 mg to 10 g of oligosaccharide component per daily dose, and from 10̂6 to 10̂12 cfu of a Bifidobacterium strain per gram of food composition.

Optionally, the food product comprises added nutrients selected from minerals, vitamins, amino-acids, unsaturated fatty acids, polyphenols, plant sterols, and mixtures thereof. For example, the food composition is an infant cereal product, a dry cereal mix, a preparation for porridge, a breakfast cereal product, a powdered diet product, a cereal bar, a powdered beverage, a milk based product or a pet food.

Preferably, the food composition is for use in the establishment and/or the maintenance of a healthy gut environment. Establishment and/or maintenance of a healthy gut environment may comprise modulation of the acidity in the colon, modulation of the short-chain fatty acids concentration in the colon, and modulation of the gut microflora, or a combination thereof.

### EXAMPLES

### Example 1

### Synergistic Effect of the Synbiotic Composition

Caco-2 is an epithelial cell line derived from human colorectal adenocarcinoma. The Caco-2/PBMC co-culture system is used as an in vitro model to study the interaction between exogenous microorganisms and gut. We employed this system to explore the potential synergistic effect between AXOS and B. lactis NCC2818. Caco-2 cells were purchased from ATCC. Freshly prepared human peripheral blood mononuclear cells (PBMC) were obtained from healthy donors. AXOS with an average degree of polymerization between 3 and 8, and an arabinose to xylose ratio (A/X) comprised between 0.18 to 0.30 was obtained from Fugeia NV. B. lactis NCC2818 was obtained internally. Samples were collected 24 hours after the treatment.

The immune response was evaluated by the chemokine levels in the medium of basal side. We first defined a concentration at which AXOS did not modulate the levels of chemokines in the Caco-2/PBMC system. Then we incubated AXOS at this concentration with varied amount of B. lactis NCC2818. Compared to the group treated with B. lactis alone, a higher level of chemokine would be considered as a synergistic effect between B. lactis and AXOS.

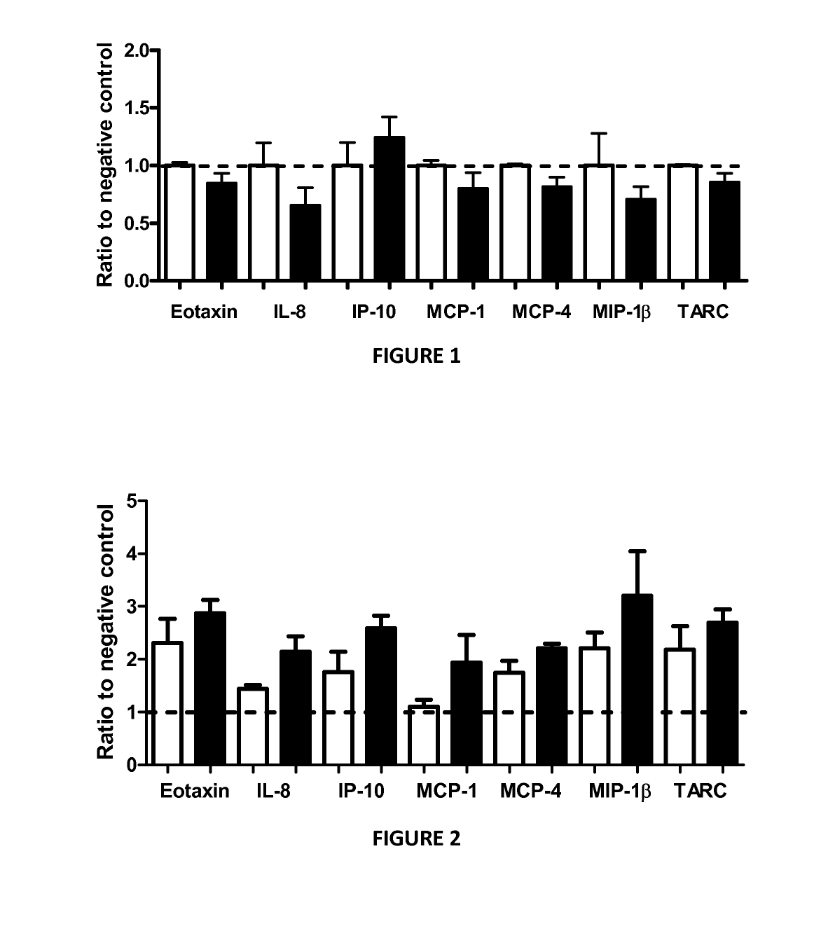

1) Selection of the AXOS concentration for evaluation. Several doses have been tested. Chemokine levels were measured using Mesoscale. We found that at 10 μg/ml level, compared to non-treatment control, AXOS had no significant impact on chemokine secretion, as shown on FIG. 1, which presents the relative chemokine concentrations as mean+SEM (SEM: standard error of the mean). The white bars represent non-treatment control. The black bars represent a 10 μg/ml AXOS treatment.

2) Evaluation of the effect of the synbiotic composition on immune response. Caco-2 cells were treated with 10̂7 CFU/ml B. lactis NCC2818 in the absence or presence of 10 μg/ml AXOS for 24 hours. Chemokine levels were measured using Mesoscale. The relative values of chemokines are shown in FIG. 2 as mean+SEM where the white bars represent B. lactis NCC2818 alone and the black bars represent B. lactis NCC2818 with AXOS. Compared to the B. lactis group, increased chemokine levels were obtained in the B. lactis+AXOS group, particularly for IL-8 and MCP-1 which are chemokines that play an important role in attracting immune cells (in particular, neutrophiles and monocytes) towards an infection site. Since 10 μg/ml of AXOS did not affect chemokine levels as described above, a synergistic effect is demonstrated between B. lactis and AXOS.

It is believed that the synergistic combination of Bifidobacterium, especially B. lactis NCC 2818, with the oligosaccharide component comprising AXOS, may modulate chemokine secretion in the colon.

### Example 2

### In-Vitro Dynamic Colon Model

An in vitro dynamic colon installation model SHIME™ (Simulator of Human Intestinal Microbial Ecosystem) operated by ProDigest was used for this experiment. The installation comprises successive reactors each representing a compartment of the digestive tract, where inoculum preparations, retention time, pH conditions, temperature, setting, gastric fluid, pancreatic and acid bile liquids in the different reactors are controlled in order to mimic in vivo conditions as closely as possible. For instance, pH is adjusted automatically by addition of a sodium hydroxide or hydrochloric acid solution into the respective reactor, depending on the target pH. Fluids from a reactor are pumped to the next. The last three reactors of the installation represent the ascending, transverse and descending colon respectively (Possemiers et al., 2004). On day 1, the installation was inoculated with feces from a 1.5 year old child. B. lactis was not detected in the inoculum. The installation was allowed to function during 2 weeks for stabilisation (stabilisation period). Then a standard diet was introduced into the installation for 2 weeks (control period) followed by test diets during 3 weeks (test period). Then a 2 weeks wash-out period was completed with a standard diet. Three test diets were assayed in this experiment: PRE with 2.5 g/day AXOS, PRO with 2.8×10̂9 cfu/day of B. lactis NCC 2818, and SYN which combines PRE and PRO: 2.5 g/day AXOS and 2.8×10̂9 cfu/day of B. lactis NCC 2818. During each period, the diet was introduced daily into the SHIME™ system.

During a treatment, when bacteria adapt to the test diet, they may produce increased amounts of short-chain fatty acids (SCFA). As a results, the environment in the reactors may acidify, which leads to addition of a sodium hydroxide solution, in order to adjust the pH in the respective reactor. Conversely, an alkalinisation of the environment in the reactors leads to an addition of a hydrochloric acid solution. In this context, the degree of acidification during the experiment can be used as a measure of the intensity of bacterial metabolism of the test diet, especially, the prebiotic blend.

The short-chain fatty acid (SCFA) concentrations and acid and base consumption were measured during the control period and the test period in the three reactors representing the colon (ascending, transverse and descending colon) for the three test diets PRE, PRO and SYN.

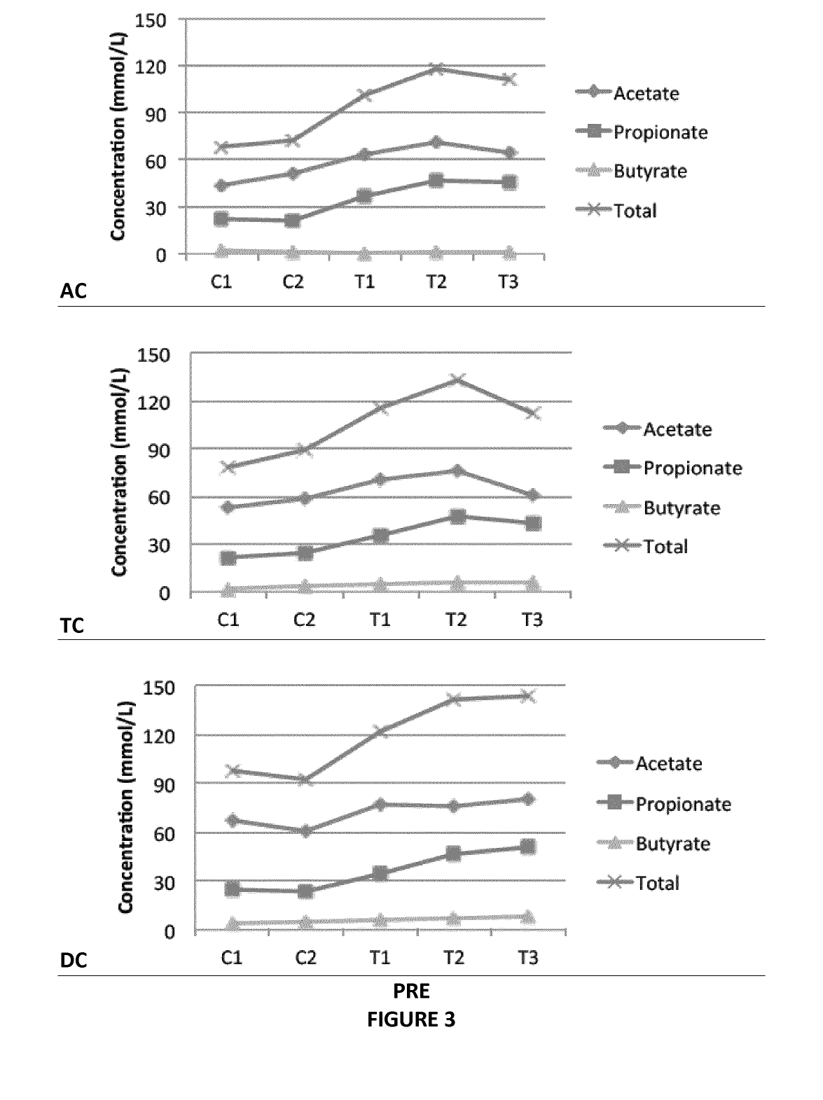

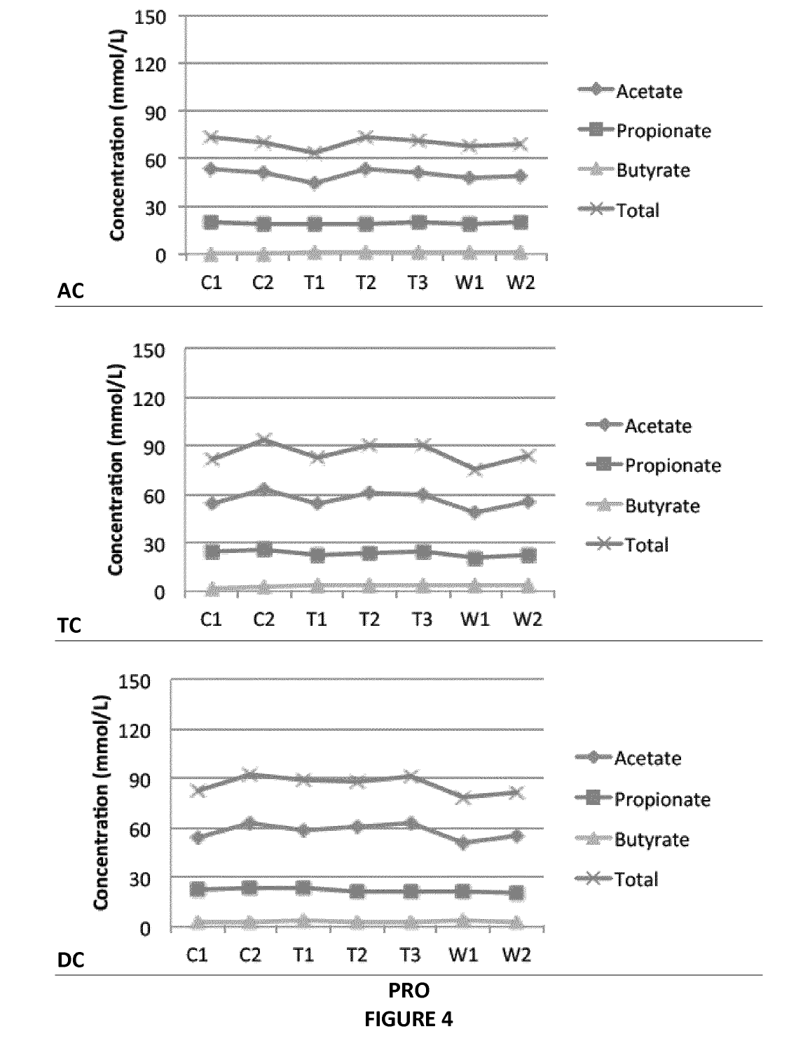

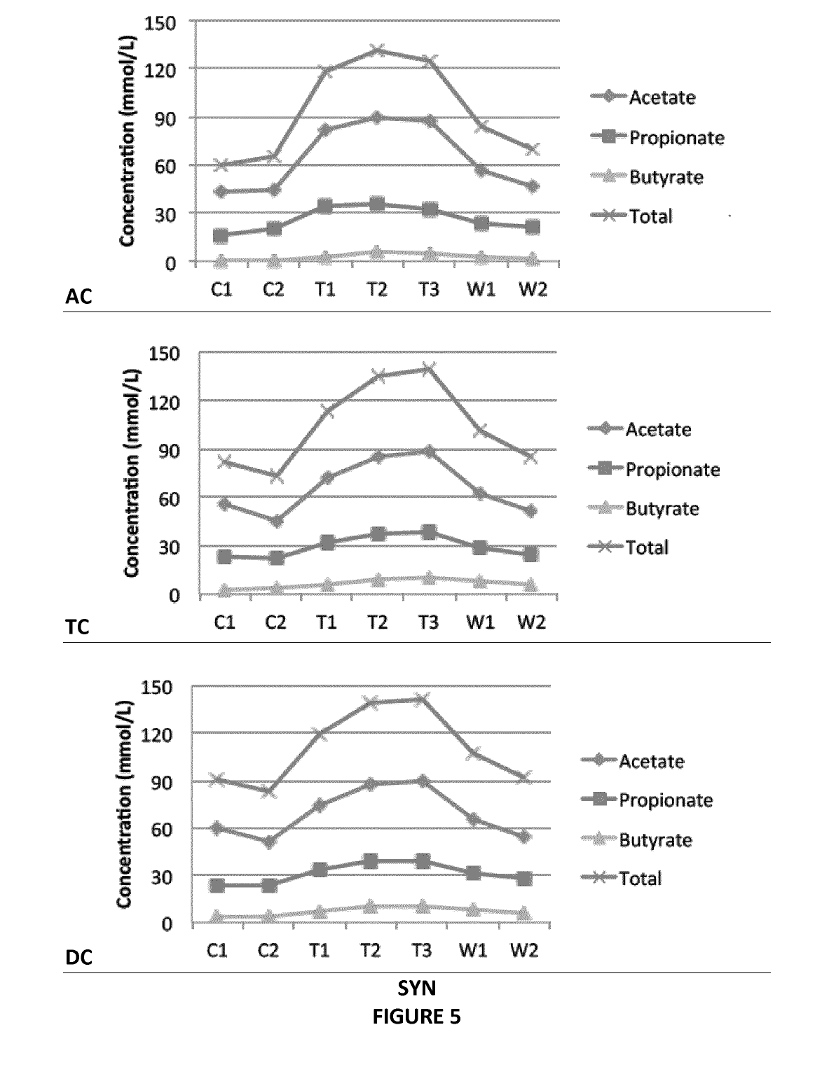

FIGS. 3, 4 and 5 show the evolution of the SCFA concentration in the ascending (AC), transverse (TC) and descending colon (DC), for diets PRE (FIG. 3), PRO (FIG. 4) and SYN (FIG. 5) respectively. Triangle: butyrate; Squares: propionate; Diamonds: acetate; X: total SCFA. C1, C2: control weeks 1 and 2. T1, T2, T3: test weeks 1, 2 and 3.

The results show that the prebiotic treatment alone induced an increase in the total SCFA concentration, which indicates that the product is well fermented in the gastrointestinal tract. The prebiotic treatment led to a higher production of propionate and acetate in all colon vessels. The combination of the prebiotic and probiotic treatment led to an increase of all the 3 main SCFAs.

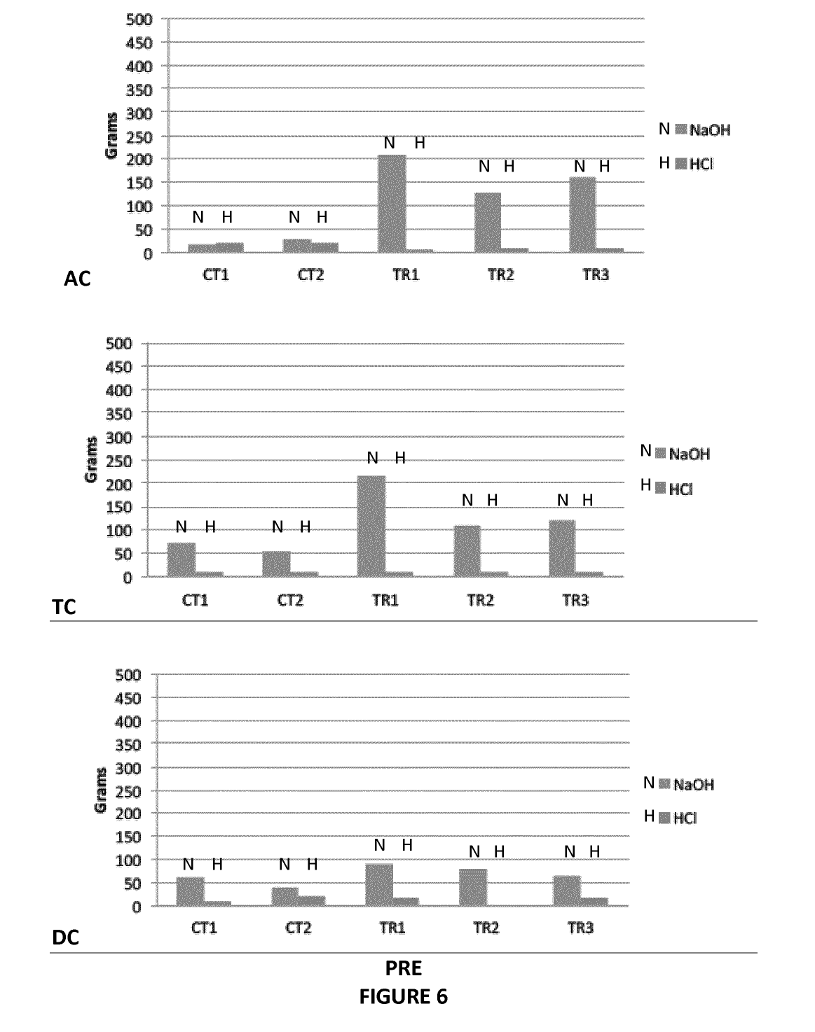

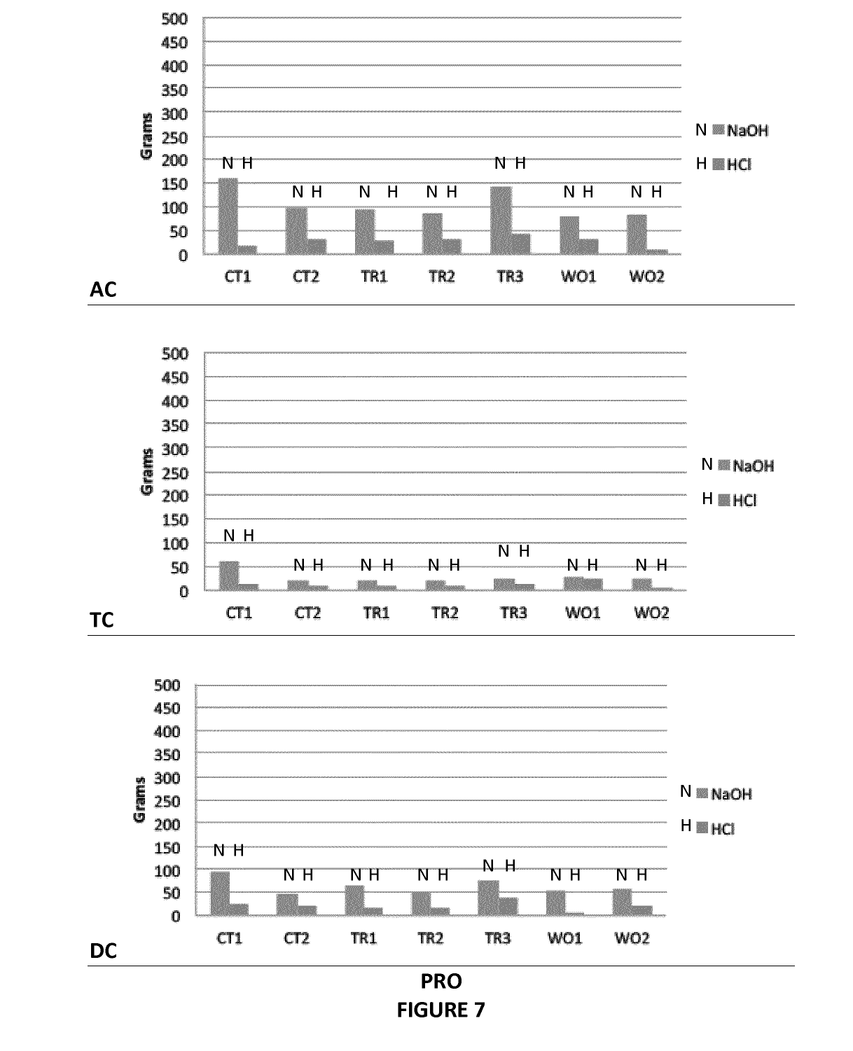

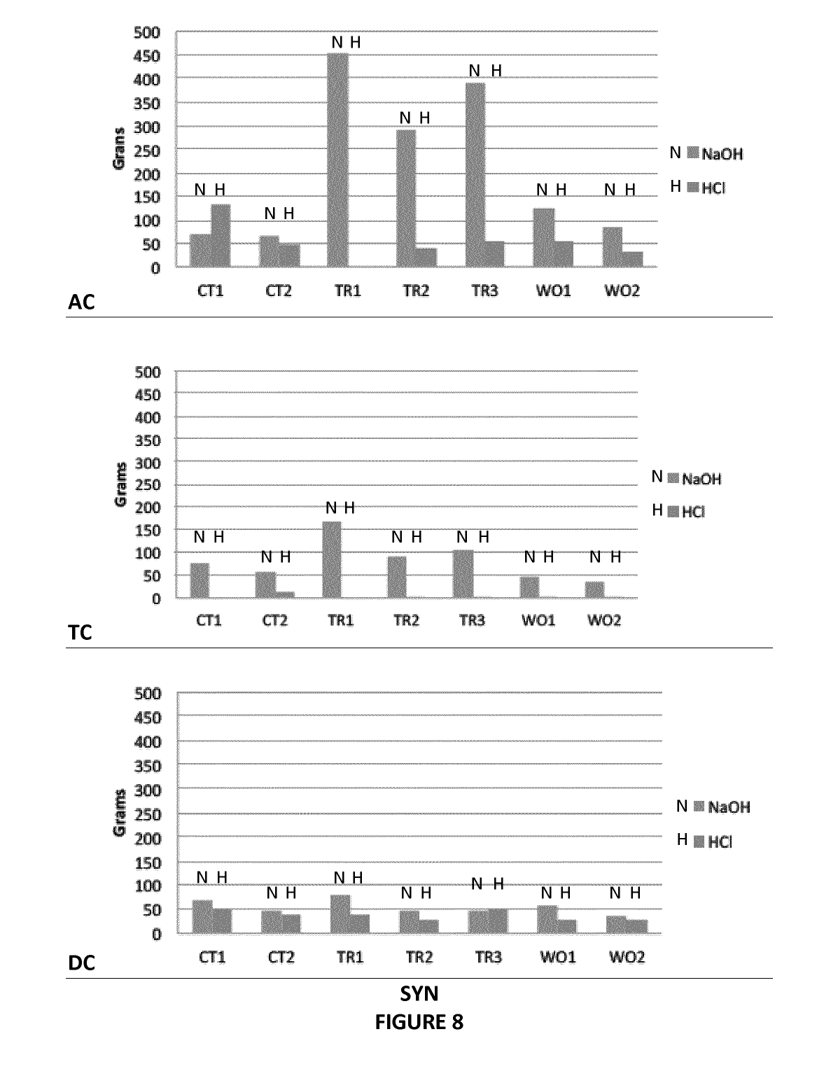

FIGS. 6, 7 and 8 show the analysis of acid base consumption as consumption of NaOH (N) and HCl (H) in the different colon regions ascending (AC), transverse (TR), and descending (DC) throughout the course of the experiment with the prebiotic (PRE), probiotic (PRO) or synbiotic (SYN) treatments.

The administration of the synbiotic induced the strongest acidification among the three test diets in the AC compartment. The prebiotic dosed alone induced a more gradual fermentation with a residual acidification still occurring in the distal colon (TC+DC).

It is believed that the synergistic combination of Bifidobacterium, especially B. lactis NCC 2818, with the oligosaccharide component comprising AXOS, may modulate the acidity in the colon, and may modulate the short-chain fatty acids concentration in the colon. Production of short-chain fatty acids is indicia of a healthy gut environment. Modulation of the acidity in the colon, especially by maintaining an acid pH in the colon, helps establishing an environment in the colon which is favourable for non-pathogenic micro-organisms, and which is not favourable for pathogenic micro-organisms, such as Salmonella (Example 3 and 4).

### Example 3

### B. lactis Growth

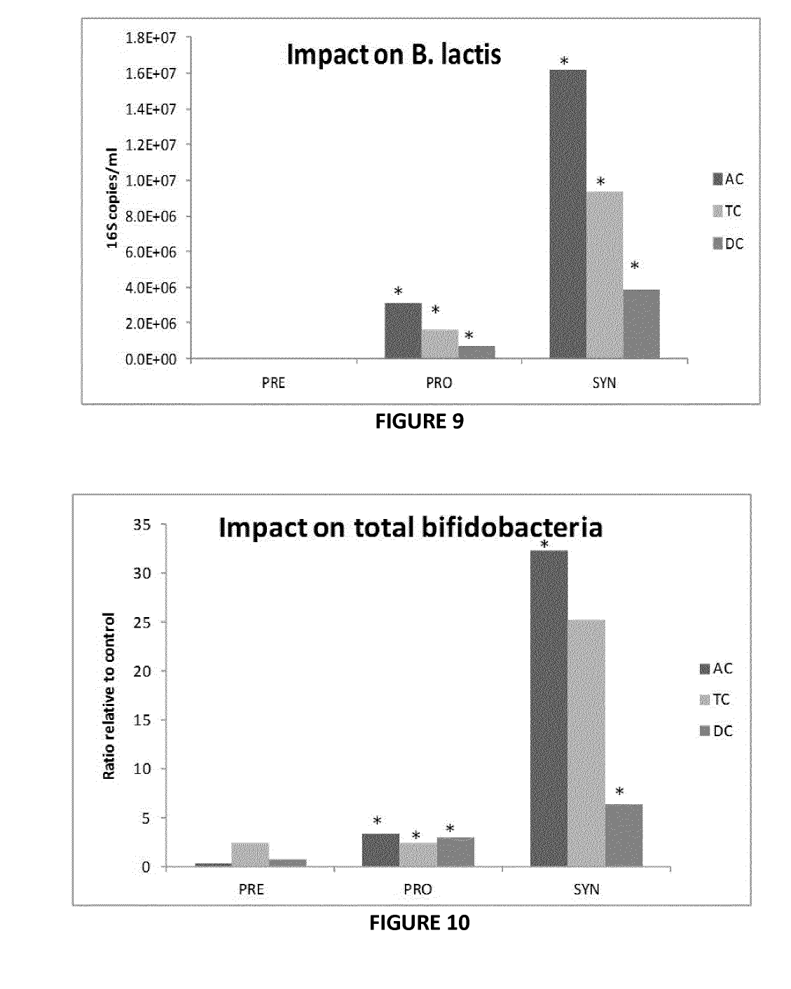

In addition, the number of B. lactis 16S copies were measured in the colonic compartments in the experiment described in Example 2. The cumulative numbers over the 3-week test periods are shown in FIG. 9. Similarly, the total Bifidobacteria population were assessed. Ratios relative to the control week populations are shown in FIG. 10.

During the probiotic treatment B. lactis was able to colonize the different areas of the colon. The combination of the probiotic with the prebiotic, led to a higher concentration of B. lactis in all compartments both during the treatment period.

It is believed that the synergistic combination of Bifidobacterium, especially B. lactis NCC 2818, with the oligosaccharide component comprising AXOS, may modulate the gut microflora, in a manner that contributes to the establishment and/or the maintenance of a healthy gut environment.

### Example 4

### Inhibition of Caco-2 Cells Infection by Salmonella

The ability of the different treatments to inhibit the invasion of gut epithelial cells by Salmonella was investigated in vitro using the Caco-2 model. This was investigated both on differentiated Caco-2 cells and on differentiated polarized Caco-2 cells. To prepare differentiated Caco-2 cells, Caco-2 cells were cultured on a cell culture plate until a tight cell monolayer was formed on the surface of the plate. To prepare differentiated polarized Caco-2 cells, Caco-2 cells were cultured in transwell inserts until a tight cell monolayer was formed and Caco-2 cells display an apical and baso-lateral polarisation. Then, the Caco-2 cells were incubated with the prebiotic, probiotic and synbiotic treatment prior to the challenge with the pathogen. After 1 hour incubation the cells were washed 3 times with PBS buffer to eliminate non adhering pathogen and incubated 1 h with gentamicin 100 mg/ml and then lysed in water during 1 hour. The pathogen released was then plated onto Petri dishes to determine the cell count. Results are expressed as cell count of internalized pathogen relative to the cell count obtained with no treatment.

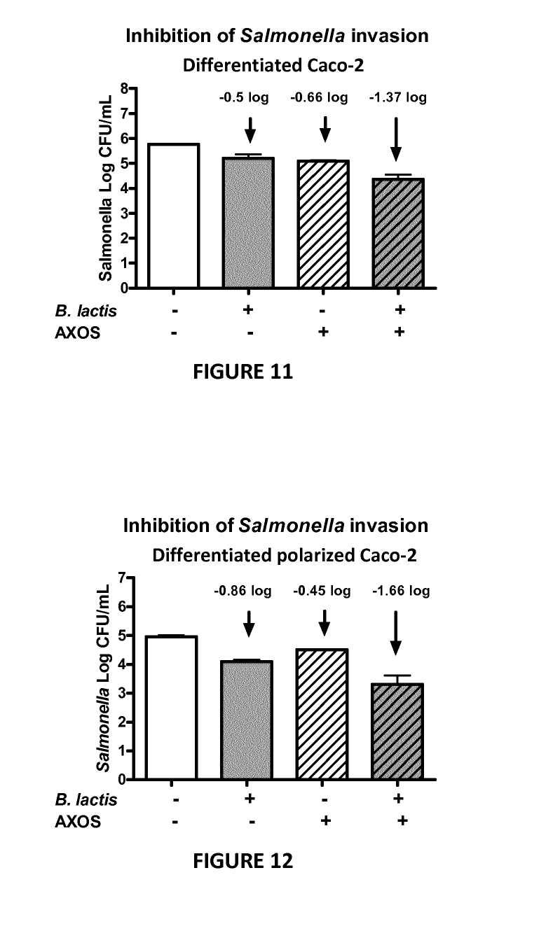

As shown on FIG. 11, the combination of the probiotic and prebiotic enables a further decrease of Salmonella invasion of differentiated CaCo-2 cells as compared to the probiotic or prebiotic treatments alone. As shown on FIG. 12, the combination of the probiotic and prebiotic enables an even greater decrease of Salmonella invasion of differentiated polarised CaCo-2 cells as compared to the probiotic or prebiotic treatments alone, and as compared to the effect on differentiated Caco-2 cells.

As shown in FIGS. 11 and 12, the effect of the combined probiotic and prebiotic is greater than the individual effect of the probiotic or prebiotic. This is even more marked in the differentiated polarised Caco-2 cells, than in the differentiated Caco-2 cells. It is believed that the synergistic combination of Bifidobacterium, especially B. lactis NCC 2818, with the oligosaccharide component comprising AXOS, may modulate the gut microflora by inhibiting pathogen infection, for instance by inhibiting the invasion of gut epithelial cells by Salmonella, in a manner that contributes to the establishment and/or the maintenance of a healthy gut environment.

### Example 5

### Infant Cereal Product

A commercial infant cereal product was obtained from Nestlé Nutrition. A composition according to the invention can be prepared by dry mixing B. lactis NCC 2818 powder and the oligosaccharide component comprising AXOS into said commercial infant cereal product, so that the final product contains from 0.01% to 0.02% by weight (dry matter) of B. lactis NCC 2818 powder, and 1.0% to 3.5% by weight (dry matter) oligosaccharide component comprising AXOS.

Although preferred embodiments have been disclosed in the description with reference to specific examples, it will be recognised that the invention is not limited to the preferred embodiments. Various modifications may become apparent to those of ordinary skill in the art and may be acquired from practice of the invention. It will be understood that the materials used and the chemical details may be slightly different or modified from the descriptions without departing from the methods and compositions disclosed and taught by the present invention.

### REFERENCE LIST

Broekaert W F, Courtin C M, Verbeke K, Van de Wiele T, Verstraete W, Delcour J A. Prebiotic and other health-related effects of cereal-derived arabinoxylans, arabinoxylan-oligosaccharides, and xylooligosaccharides. Crit Rev Food Sci Nutr. 2011 February; 51:178-94.
Crittenden R G, Morris L F, Harvey M L, Tran L T, Mitchell H L, Playne M J. Selection of a Bifidobacterium strain to complement resistant starch in a synbiotic yoghurt. J Appl Microbiol. 2001; 90(2):268-78.
Eeckhaut V, Van I F, Dewulf J, Pasmans F, Haesebrouck F, Ducatelle R, Courtin C M, Delcour J A, Broekaert W F. Arabinoxylooligosaccharides from wheat bran inhibit Salmonella colonization in broiler chickens. Poult Sci. 2008 November; 87:2329-34.
Francois I E, Lescroart O, Veraverbeke W S, Marzorati M, Possemiers S, Evenepoel P, Hamer H, Houben E, Windey K, et al. Effects of a wheat bran extract containing arabinoxylan oligosaccharides on gastrointestinal health parameters in healthy adult human volunteers: a double-blind, randomised, placebo-controlled, cross-over trial. Br J Nutr. 2012 Feb. 28; 1-14.
Grootaert C, Van den Abbeele P, Marzorati M, Broekaert W F, Courtin C M, Delcour J A, Verstraete W, Van de Wiele T. Comparison of prebiotic effects of arabinoxylan oligosaccharides and inulin in a simulator of the human intestinal microbial ecosystem. FEMS Microbiol Ecol. 2009 August; 69:231-42.
Komiyama Y, Andoh A, Fujiwara D, Ohmae H, Araki Y, Fujiyama Y, Mitsuyama K, Kanauchi O. New prebiotics from rice bran ameliorate inflammation in murine colitis models through the modulation of intestinal homeostasis and the mucosal immune system. Scand J Gastroenterol. 2011 January; 46:40-52.
Mercenier A, Foligné B, Dennin G, Goudercourt D, Pot B, Rochat F. Selection of candidate probiotic strains protecting agains murine acute colitisty and new ways for prevention of infections. J Pediatr Gastroenterol Nutr. 3 May 2006; 43(5):E38
Ogawa K, Takeuchi M, Nakamura N. Immunological effects of partially hydrolyzed arabinoxylan from corn husk in mice. Biosci Biotechnol Biochem. 2005 January; 69:19-25.
Possemiers, S. et al. PCR-DGGE-based quantification of stability of the microbial community in a simulator of the human intestinal microbial ecosystem. FEMS Microbiology Ecology. 2004; 4: 495-507.

1. A method for the establishment and/or the maintenance of a healthy gut environment comprising administering to an individual in need of same a composition comprising a probiotic micro-organism comprising a Bifidobacterium strain, and an oligosaccharide component comprising: from 30 to 40% by weight of arabino-xylo-oligosaccharides having an average degree of polymerization from 2 to 50, and from 35 to 45% by weight of xylo-oligosaccharides having a degree of polymerization ranging from 2 to 9.

2. The method according to claim 1, wherein establishment and/or the maintenance of a healthy gut environment comprises modulation of the acidity in the colon, modulation of the short-chain fatty acids concentration in the colon, and modulation of the gut microflora.

3. The method according to claim 1, wherein the Bifidobacterium strain is selected from the group consisting of Bifidobacterium longum, Bifidobacterium lactis, Bifidobacterium animalis, Bifidobacterium breve, Bifidobacterium infantis, Bifidobacterium adolescentis, and mixtures thereof.

4. The method according to claim 1, wherein the Bifidobacterium strain is selected from the group consisting of Bifidobacterium longum NCC 3001, Bifidobacterium longum NCC 2705, Bifidobacterium breve NCC 2950, Bifidobacterium lactis NCC 2818, and mixtures thereof.

5. The method according to claim 1, wherein the arabino-xylo-oligosaccharides (AXOS) has an average degree of polymerisation (DP) of from 2 to 15, and an arabinose to xylose ratio (A/X ratio) of between 0.18 to 0.30.

6. The method according to claim 1, wherein a ferulic acid residue is bound to the arabino-xylo-oligosaccharides (AXOS), via an ester linkage, preferably to an arabinose residue.

7. The method according to claim 1, wherein the oligosaccharide component comprises beta-glucan, xylo-oligosaccharides, xylobiose, and mixtures thereof.

8. A synbiotic composition comprising a probiotic micro-organism comprising a Bifidobacterium strain, and an oligosaccharide component comprising arabino-xylo-oligo saccharides.

9. The synbiotic composition according to claim 8, wherein the Bifidobacterium strain is a Bifidobacterium lactis strain.

10. The synbiotic composition according to claim 8, wherein the arabino-xylo-oligosaccharides is derived from cereal.

11. A food composition comprising a synbiotic composition comprising a probiotic micro-organism comprising a Bifidobacterium strain, and an oligosaccharide component comprising arabino-xylo-oligosaccharides.

12. The food composition according to claim 11, wherein the food composition is selected from the group consisting of infant cereal products, dry cereal mixes, preparations for porridge, breakfast cereal products, powdered diet products, cereal bars, powdered beverages, milk based products, and pet-food.

13. The food composition according to claim 11, wherein the food composition comprises:
from 100 mg to 10 g of oligosaccharide component per daily dose, and
from 10̂6 to 10̂12 cfu of a Bifidobacterium strain per gram of food composition.

14. The food composition according to claim 13, wherein the Bifidobacterium strain is a Bifidobacterium lactis strain.

15. (canceled)
